(19) United States
(12) Patent Application Publication
(10) Pub. No.: US 2024/0000413 A1
(43) Pub. Date: Jan. 4, 2024

(54) PRESSURE AND X-RAY IMAGE PREDICTION OF BALLOON INFLATION EVENTS

(71) Applicant: KONINKLIJKE PHILIPS N.V., Eindhoven (NL)

(72) Inventors: MOLLY LARA FLEXMAN, MELROSE, MA (US); GRZEGORZ ANDRZEJ TOPOREK, CAMBRIDGE, MA (US); JOCHEN KRUECKER, ANDOVER, MA (US); ASHISH SATTYAVRAT PANSE, BURLINGTON, MA (US)

(21) Appl. No.: 18/037,668

(22) PCT Filed: Dec. 1, 2021
(86) PCT No.: PCT/EP2021/083759

§ 371 Date: May 18, 2023

Related U.S. Application Data
(60) Provisional application No. 63/121,289, filed on Dec. 4, 2020.

(51) Int. Cl. A61B 6/00; A61B 6/12; A61M 25/10; G16H 30/40; G16H 30/20; G16H 50/30; G06N 20/00

(52) U.S. Cl. CPC A61B 6/5211; A61B 6/462; A61B 6/12; A61M 25/104; G16H 30/40; G16H 30/20; G16H 50/30; G06N 20/00; A61B 2562/0247

(57) ABSTRACT

System and related method for supporting a balloon catheter (BC) procedure. The system comprises an input interface (IN) for receiving input data. The input data comprises i) image data acquired of a balloon catheter in a vessel of a patient (PAT), and ii) one or more pressure readings collected by a pressure sensor (S) of the balloon catheter (BC). A trained machine learning module (MLM) is configured to predict, based on the input data, a prediction result including an event in relation to i) the balloon catheter and/or ii) a section of a vessel in which the balloon catheter is residable.

### FIELD OF THE INVENTION

The invention relates to a system for supporting an intervention, to a related method, to a system for training a machine learning module, to a related machine learning training method, to a computer program element, and to a computer readable medium.

### BACKGROUND OF THE INVENTION

Balloon angioplasty, also known as percutaneous transluminal angioplasty (PTA), is one of the most frequently used procedures in managing atherosclerotic plaque. Atherosclerosis of peripheral arteries causes peripheral artery disease, which affects approximately 8 million people in the USA alone. Typically, a balloon catheter is passed over a guide wire to the site of the lesion and is then inflated using a (mix of) contrast substance/agent and/or other fluid that is pumped into a balloon portion of the balloon catheter to cause an increase in internal pressure in the balloon portion. The inflating balloon portion can thus force an existing stenosis in a blood vessel wider and/or open. Balloon angioplasty is also commonly used in conjunction with other therapies (either before or after) such as stenting and laser atherectomy, and other.

The rate of restenosis following therapy is relatively high in the months and years following treatment leading to 20% of patients receiving repeat procedures. Studies have shown that restenosis is associated with excessive repair action of for example an artery due to mechanical damage caused during the treatment—for example, by overstretching of the arterial wall. Simulations on this topic have shown that damage is higher to certain regions of the lesion—specifically those that are not protected by a layer of calcification. The same study found that softening plaque (for example, via ballooning) could lead to lower peak stress on the vessel wall, and thus to less damage.

Recently, drug coated balloons have shown promising results in reducing restenosis and hence need for re-interventions. These balloon catheters include a coating of a drug, for example Paclitaxel, that acts to inhibit arterial smooth muscle cell proliferation in the repair action. Usage instructions (IFU) for some drug coated balloon catheters indicate that for proper drug delivery to the target lesion, inflation the balloon should be maintained for a certain period of time, such as a for a minimum of 60 seconds for example. There is some evidence that prolonged inflation times during plain balloon angioplasty can have improved outcomes.

During inflation of the balloon, there are certain adverse events that can happen. It is advantageous to prevent adverse events since they can impact the patient and procedure, but it may also be beneficial to log those events and report the data to a regulator for example, such as the FDA (Food and Drug Administration), a US federal regulatory agency. In addition, there is a desire from manufacturers to have appropriate data collected around those events. Such adverse events include balloon rupture, twisting or fold-over that prevents inflation or deflation of the balloon portion.

Currently, the medical user is responsible for integrating/interpreting fluoroscopy image feedback together with the pressure information received from the balloon catheter's inflation gauge.

### SUMMARY OF THE INVENTION

There may therefore be a need for improved support for, in particular, medical interventions.

An object of the present invention is achieved by the subject matter of the independent claims where further embodiments are incorporated in the dependent claims. It should be noted that the following described aspect of the invention equally applies to the said related method, to the system for training a machine learning module, to the said related training method, to the computer program element, and to the computer readable medium.

According to a first aspect of the invention there is provided a system for supporting a balloon catheter procedure, comprising:
at least one input interface for receiving input data, comprising i) image data acquired of a balloon catheter in a vessel of a patient, and ii) one or more pressure readings collected by a pressure sensor of the balloon catheter;
a trained machine learning module configured to predict, based on the input data, a prediction result including an event in relation to i) the balloon catheter and/or ii) a section of a vessel in which the balloon catheter is residable.

The input data may be received as part of one more data streams, such as in video feed (and/or a pressure measurements feed. Video feeds may be generated in certain imaging modalities, such as in fluoroscopy or others modalities. However, processing of data streams is not a requirement herein.

In one embodiment, at least one of the input data i), ii) is obtained by a sampler of the system sampling a respective original data stream, wherein a sampling frequency of the said sampler is variable. The sampling frequency may vary. In particular, the sampling frequency may depend on an earlier prediction result, in particular when the input data is received as part of such as one or more data streams as mentioned earlier.

In one embodiment, the system comprises a reporting module configured to report the prediction result numerically and/or graphically.

In one embodiment, the reporting module includes a visualizer component configure to produce a graphics display for displaying the prediction result on a display device.

In one embodiment, the graphics display further includes at least some of the image data and/or at least some of the pressure readings.

In one embodiment, the reporting module is operable to annotate at least some of the received image data with said prediction result. The annotation may be fully automatic or may rely at least in parts on user input, as through a suitable user interface (keyboard, touchscreen, pointer tool) etc.

In one embodiment, wherein, based on the prediction result, at least some of the image data and/or some of the pressure readings are stored in a memory.

In one embodiment, the machine learning module includes a trained machine learning model of the neural network type.

In one embodiment, the machine learning model includes at least one convolutional layer and/or at least one recurrent layer.

In one embodiment, the event is a rupture or twisting of a balloon portion of the balloon catheter, and/or stress on the vessel, caused by an inflation of the balloon.

In another aspect there is provided a training system configured to train, based on training data, the said machine learning module of the system as per any one of the previous embodiments.

In another aspect there is provided a method of supporting a balloon catheter procedure, comprising:
receiving input data comprising i) image data acquired of a balloon catheter in a vessel of a patient and ii) one or more pressure readings collected by a pressure sensor of the balloon catheter; and
processing the input data by a trained machine learning module to obtain a prediction result for an event in relation to i) the balloon catheter and/or ii) a section of a vessel in which the balloon catheter is residable.

In yet another aspect there is provided a method of training, based on training data, the said machine learning module of the system as per any one of the previous embodiments mentioned above.

Using machine learning technology to process a combination of those two inputs (imagery and pressure reading(s)) allows capturing interaction between the balloon and the anatomy that can be used to predict stress on the vessel and adverse events. Machine-learning processing of both data feeds, imagery and pressure readings, allows reliable and robust prediction, and thus possibly prevention of adverse events such as malfunctioning of the medical device, such as of a balloon catheter. Adverse events may include balloon rupture, fold-over or twisting that prevents inflation or deflation. It has been found that using either data feed on its own is less desirable. For example, calcifications in the vessel walls may include sharp crystal formation that may cause rupture even for modest pressure readings. Also, imagery on its own may not capture “the whole picture” as it were. The balloon inflation may look “good” in the image feed, but in fact the internal pressure within the balloon is about to exceed its rating, etc. The event of interest may also be formulated as a predefined maximally allowed level of mechanical stress exerted by the balloon on a certain section of the vessel's wall.

The system also allows reliably logging/reporting the evens if they do indeed occur, thus providing valuable feedback to manufacturers, regulators or other parties.

Whilst processing of a single image or pressure reading datum may be sufficient in some instances, processing two data streams of image frames and/or pressure readings, may be preferred in embodiments to improve performance.

In another aspect there is provided a computer program element, which, when being executed by at least one processing unit, is adapted to cause the processing unit to perform the method as per any one of the above mentioned embodiments.

In another aspect still, there is provided a computer readable medium having stored thereon the program element.

The proposed system method, arrangement computer program element and computer readable medium can be used with mobile or fixed interventional x-ray systems. It can used for substantially all balloon catheter types, including specific relevance drug-coated angioplasty balloon catheters, scoring balloon catheters, bridge occlusion balloon catheters and others.

“User” relates to a person, such as medical personnel or other, operating the imaging apparatus and/or overseeing the imaging procedure and/or medical intervention. In other words, the user as understood herein is in general not the patient.

“Image data” as used herein includes raw or processed imagery as provided by the imaging apparatus. However, “image data” also includes parts of the original imagery, such as information extracted from the imagery via segmentation for example. The format of the image data may be matricial such as 2D, 3D or 4D (with one dimension for time as in video feeds for 2D or 3D native image data), but may also include instead or in addition other suitable functional or parametric descriptions such as segmentations. The suitable functional or parametric descriptions may include data structures that indicate or refer to shape, locale, extent etc., to so define a segmentation in image domain. A bit mask may be used for example.

In general, “machine learning” includes a computerized arrangement/module, that implements a machine learning (“ML”) algorithm. In model-based machine learning, a machine learning model is adapted to perform a task. This adaption is called “training. Task performance by the ML model or ML module improves measurably, the more (new) training data is used to train the model. The performance may be measured by objective tests applied to output generated by ML system when processing test data. The performance may be defined in terms of a certain error rate to be achieved for the given test data. See for example, T. M Mitchell, “Machine Learning”, page 2, section 1.1, McGraw-Hill, 1997.

### BRIEF DESCRIPTION OF THE DRAWINGS

Exemplary embodiments of the invention will now be described with reference to the following drawings, which, unless stated otherwise, are not to scale, wherein:

FIG. 1 shows a schematic block diagram of an arrangement to support a medical intervention;

FIG. 2 shows details of a balloon catheter;

FIG. 3 shows a projection image of a balloon catheter;

FIG. 4 shows a block diagram of a computerized system to support a medical intervention;

FIG. 5 shows a series of pressure readings and a series of images;

FIG. 6 shows annotations of imagery as may be produced by the computerized system for supporting interventions;

FIG. 7 shows a flow chart of a computer implemented method of supporting a medical intervention;

FIG. 8 shows a schematic block diagram of a machine learning model;

FIG. 9 shows a block diagram of a computer implemented training system for training a machine learning model; and

FIG. 10 shows a flow chart of a computer implemented method of training a machine learning model.

### DETAILED DESCRIPTION OF EMBODIMENTS

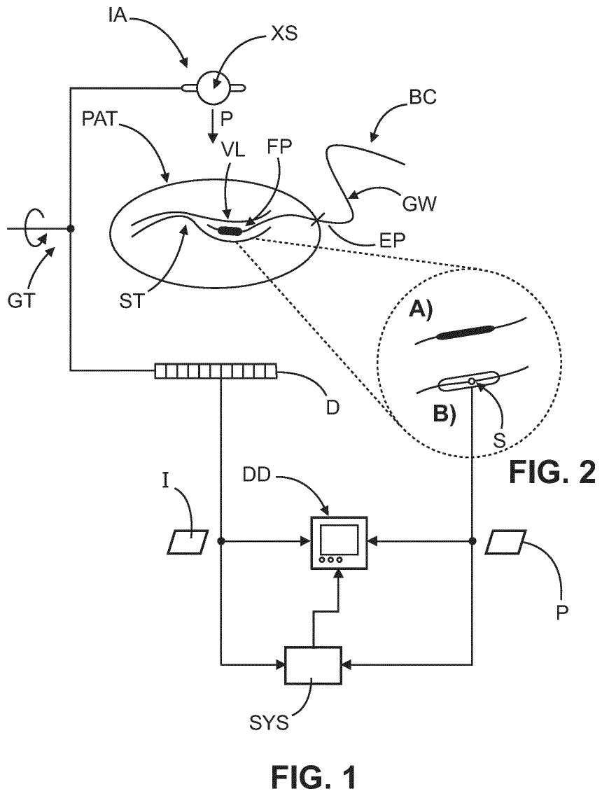

With reference to FIG. 1, this shows a schematic block diagram of a computerized system SYS configured to support an intervention performed in relation to a human or animal patient PAT.

The intervention is image-guided in that it is performed during operation of an imaging apparatus IA. The imaging apparatus IA is preferably an x-ray imaging apparatus including an x-ray source XS and a detector D, but other imaging modalities such as MRI, ultrasound or optical OCT and others still are also envisaged herein. The imaging apparatus mainly envisaged herein generates projection imagery.

As will be explained more fully below, the system SYS is arranged on one or more computing systems to processes data generated during the intervention. More particularly, imagery I generated by the imaging apparatus IA and pressure readings P acquired by an in-situ sensor S (to be discussed in more detail below) are processed together by the system SYS to predict one or more of i) a medical outcome in relation to the patient undergoing the intervention and ii) certain events of interest, such as adverse events. Such events may occur in relation to the patient and/or the medical equipment used in the intervention.

The medical equipment mainly envisaged herein may include a balloon catheter BC. Such catheters BC may be used in certain interventions such as angioplasty interventions. Such angioplasty interventions may be used to treat a stenosis. One such stenosis type may present in a mammal's (human or animal) vessel in the veinous or arterial system. Cardiac stenosis in the coronaries of the human heart is one example mainly envisaged herein, but other applications are not excluded. Stenosis may cause a range of medical complications due to undersupply of oxygen caused by poor blood circulation.

Broadly, and with particular reference to cardiac stenosis, this may be treated by advancing a distal portion of the balloon catheter through the patient to the lesioned site such as the strictured site ST, a portion of a lesioned blood vessel. A guide wire GW may be used to navigate and push the cathether's distal treatment portion to the site ST. A range of different types of balloon catheters are envisaged herein including those of the over-the-wire (OTW) type or rapid exchange (RX) type, or others still. In general, balloon catheters BC includes a tube with the said distal end, and a proximal end. The proximal end remains outside the patient with a user, such as an interventional cardiologist, to control the operation of the catheter. The distal portion is introduced into the patient through an entry point EP such as at the femoral artery or vein and is advanced through the vasculature to the lesioned site ST. The distal portion of the catheter tube in balloon catheters is formed into a balloon portion FP that is inflatable with liquid such as water or preferably a saline solution. The liquid is administered through a port portion at the proximal end to inflate the balloon portion FP. Upon removal of some or all of the liquid, the balloon deflates. The balloon portion FP is formed from an elastic material such as an elastomer with suitable elasticity properties. The balloon portion FP is advanced by use of the guide wire via the entry point EP to the lesioned site and during operation of the image apparatus IA to assist navigation through the vasculature. Upon arrival at the lesioned site ST, the balloon portion FP may be inflated by introduction of the liquid into the balloon FP, thus causing a force on the vessel VL walls to be exerted to so broaden the stricture thus treating same. Treatment of the stenosis as described above is mainly envisaged herein, however, so are other treatment options such as the placement of a stent for example. Whilst cardiological interventions are mainly envisaged herein, this is not to exclude other medical interventions that use balloon catheters CB such as interventions in relation to the urinary system in urological applications for instance.

FIGS. 2A, B are close-ups that show respectively two states of the balloon portion FP of the balloon catheter BC. FIG. 2A shows the deflated state of the balloon portion which can be ascertained as the slightly thickened part (shown in black) of the distal portion of the balloon catheter, whilst FIG. 2B shows an inflated state of the balloon portion FP.

The inflation of inflatable balloon portion FP causes an increase in pressure, which can be measured by a pressure sensor S integrated into the balloon catheter BC, preferably in the balloon portion FP of the catheter BC or into an inflation unit of the balloon catheter. However, the pressure sensor S may be attached at any point along the pressure inducing pathway. For example, in some embodiments, the sensor S is attached/integrated at or in an injector/inflator pump that is operable for pressurized supply of the fluid to balloon portion FP. A processing unit (not shown) of the balloon catheter arrangement BC captures and digitizes the pressure readings P. The pressure readings may be supplemented by additional information, such a volume V readings etc. In the following we will not refer specifically to such additional information and any reference below to “pressure readings” should be construed to optionally include such additional information. Equally, the pressure readings may be supplied direct in terms of pressure quantities (such as PSI, bar etc) but may be provided instead in terms of surrogate quantities, such volume, temperature etc that directly correlate with balloon portion pressure or that can be converted to such pressure quantities. As such, the processing by the proposed system SYS may well proceed on such surrogate quantities such as volume etc, and conversion to pressure quantities is not necessarily required herein. Although we will refer to herein to “pressure readings”, any such reference should be construed herein to include reference to such surrogate quantities.

Conventionally, the treatment of the stenosis ST includes multiple phases, including positioning the balloon portion FP close to the stenosis, exerting a certain push by the user to force the balloon portion FP at least partly through or past the stenosis, applying the liquid to cause the balloon portion to inflate. The inflation effects the broadening of the stenosis and/or effects the delivery of stent, or effects any other treatment to be performed at the lesion site ST. The phases are usually performed by the user under image-guidance as mentioned. The image apparatus IA produces a life feed of x-ray images I(“frames”), at a suitable frame rate such as 60 fps for example (other frame rates may also be used). The video feed may be displayed on a display device DD as a motion picture. Although capture and display of still imagery is not excluded herein, it is in particular the video feed that allows the user to ascertain the correct unfolding of the treatment procedure, such as the positioning of the catheter, its inflation, etc. Specifically, the phases of placement, push-through and deflation and/or inflation cause interaction of the balloon catheter with the vessel VL, in particular with its wall, in the course of which the balloon portion FP assumes a range of different geometrical configurations which can be ascertained through the acquired imagery. A good number of interventions run smoothly but sometimes certain adverse effects may occur such as a rupture of the balloon portion FP (“rupture event”). This can have many causes, such as unfavorable balloon-vessel interaction or unfavorable properties of the vessel. Specifically, balloon rupture event can occur due to defects in the balloon, over-inflation of the balloon beyond the rated burst pressure (RBP). Lesion morphology such as sharp calcifications can also cause balloon rupture. In most cases balloon rupture does not cause serious complications. However, at time, it can lead to air embolism, vessel dissection/perforation, balloon fragments detached into the body, and longer procedure times. Other adverse events include twists, kinks or folds in the balloon portion FP that prevents (full) inflation to the desired extent. Such adverse events such as rupture, twisting, etc are adverse as they prevent a successful conclusion of the intervention. For example, such events may result in failure to broaden the stenosis, either not at all or not sufficiently so, or that the drug could not be deposited at the site of the lesion, etc.

In addition to the imagery provided by the apparatus, there is, as mentioned briefly above, another data feed, namely a time series of the pressure readings P acquired by the sensor S. It has been found that by observing the pressure readings in conjunction with the different geometrical configurations of the balloon portions FP as captured be the video feed imagery I, a reliable prediction can be made as to whether or not an adverse effect are imminent. The adverse effect may include the said rupture event or other event, such as a twist or fold-over events, that compromise deflation and/inflation of balloon portion FP. Other malfunction events that may compromise the intervention as also envisaged herein.

Whilst the imagery I provides clues on the geometrical configuration, the pressure readings provide an additional information layer in relation to the balloon-portion-versus-vessel interaction. The two data feeds, the imagery encoding geometrical information plus the pressure readings, may be used together to predict the outcome of the treatment procedure. The outcome may include, for instance, whether or not the patient may experience re-stenosis and thus may require repeated interventions. This may be expressed as a time-defined patency, such as patency at m months, for example m=12. Other outcomes may be related to aortic insufficiency, need for follow-up surgery, even death, especially for balloon valvuloplasty for calcific aortic stenosis.

The computerized system SYS is configured to jointly process the double-data feed, the imagery I and the pressure readings P, to compute those predictions on adverse events (“E”) and/or outcome (“O”). Preferably, the system SYS uses a machine learning module or component MLM, previously trained on suitable training data. In the course of the training, and in some embodiments, a machine learning model M of the module MLM is adapted based on the training data to configure the machine learning component. Whilst there is a correlation between such adverse events/outcome and the double-feed information encoded jointly in the imagery and the pressure reading, it has been shown to be difficult to analytically and/or explicitly model the exact relationships between those events/outcomes given the imagery and pressure readings. Such analytical modelling may also suffer from low efficiency, overfitting and poor performance. Machine learning (“ML”) approaches are beneficial in that they generalize well from the training data as they do not require an explicit modeling of the said correlation. Provided a sufficiently large set of training data is available. ML has been found to provide a well-generalized approximation of the latent relationship between outcomes/events and the double-data-feed of catheter imagery I and pressure readings P.

Referring now briefly to the imaging apparatus IA, this preferably allows acquiring imagery from different projection directions d. The imaging apparatus may include an optional gantry GT to which the x-ray source XS and the detector D are connected. The projection imagery along the different projection directions may be acquired by rotation of the gantry around the lesioned site ST, and with it the source XS and detector D. Such gantry-based imaging apparatuses include C- or U-arm systems and are mainly envisaged herein, but so are (CT) computed tomography scanners. Non-gantry based imaging solutions are also envisaged, such as mobile or portable imaging devices, where there is no, or no permanent, physical connection between detector D and radiation source XS. The imagery I processed by the system SYS may be projection imagery in projection domain as recorded by the detector D, or may comprise reconstructed imagery in image domain obtained by a computed tomography algorithm.

During imaging, the x-ray source XS emits an x-ray beam which propagates along a projection direction d to interact with patient tissue and/or the balloon catheter to cause a modified x-ray beam to emerge at the far end of the patient, and be detected at detector D. Data acquisition circuitry (not shown) of the detector D converts the received modified radiation into a set of numbers (“pixel values”), preferably stored in a respective matrix per frame, with respective rows and columns. The pixel values represent detected intensities. The pixel values per frame can be used by a visualization component VC to effect display of the image on a display device DD during the intervention.

As mentioned, preferably a series of such images is generated to cause a video feed to be displayed on the monitor device DD. Real time imaging can thus be provided, assisting in real time monitoring of the intervention. However, the provision of a series of such images is not necessarily required in all embodiments, and a single such image frame captured in conjunction with a single or more pressure readings may suffice in some circumstances for computing the prediction. However, the processing by the machine learning module MLM of plural frames and/or plural pressure readings may be more reliable and/or more robust, as this may allow the ML module MLM to take into account trends when computing the prediction.

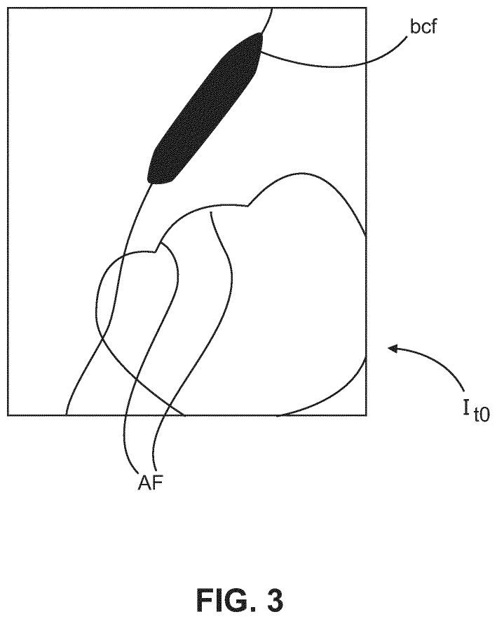

FIG. 3 is an example of such a projection image acquired by an x-ray imaging apparatus during the intervention at a certain time to. Frame at time to shows an example of a projection footprint bcf of the balloon catheter BC and certain anatomical features AF in relation to the vessel VL. The soft tissue anatomical features, such as blood vessels VL, are usually not well discernable in native x-ray imagery due to poor contrast. Angiographic techniques may be used to boost soft-tissue contrast. Angiography involves administering a contrast agent to the patient which is allowed to accumulate at the lesion site ST, and images are then taken, the contrast agent thus conferring better contrast in the recorded imagery. Digital subtraction techniques may be used to process imagery acquired before and after administration of the contrast agent to more clearly define the projection footprint of the relevant system of vessels sometime referred to as a “vessel tree”. Road mapping techniques may be used to extract by segmentation the vessel tree and superimpose same on x-ray frames acquired whilst there is no contrast agent present any more at the lesioned site, thus still providing to the user spatial information including location and extent of the vessel tree.

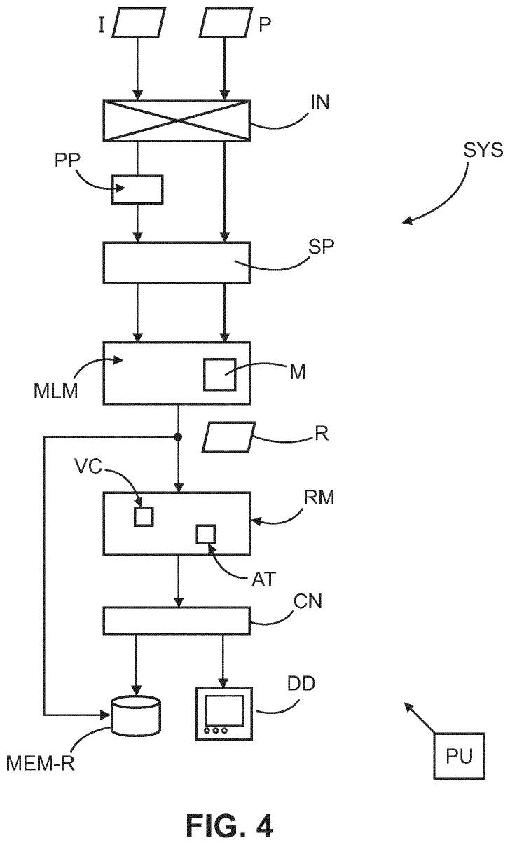

Referring now to FIG. 4, this shows a schematic block diagram of the computerized system SYS for supporting medical interventions as envisaged herein. The system may be implemented by one or more computing devices PU. A single such device may be used in alternative embodiments. If there are a plurality of such devices, these may possibly be geographically distanced, the system thus being implemented in a cloud architecture or similar. A server-based system may be used. Alternatively, the whole or part of the computerized system may be integrated in the balloon catheter and/or into the imaging apparatus. In embodiments the system is wholly or partly integrated into a work station, that is, a computing unit communicatively coupled to the imaging apparatus. The computerized system may be implemented wholly or partly as software or hardware, or as a combination both.

Broadly, and with continued reference to FIG. 4, the system SYS includes one or more data interfaces IN through which the image feed I and the pressure reading feed P are received, not necessarily at the same time. Time stamping may be used to pair up the readings with frames. The input data comprising imagery and pressure readings may be received direct from the imager/balloon catheter, or the input data may be stored/buffered in storage and may then be requested or pushed therefrom to be received at input(s) IN. The storage/buffer may be part of the imaging system IA.

Optionally, an image pre-processor PP may be used to process the received imagery to extract, by segmentation or other techniques, relevant image structures such as the balloon catheter footprint BCF and/or the vessel tree etc. Whilst the whole feed of images or pressure readings can be processed by the system, in order to boost responsiveness and/or to save computation resources, it may be beneficial to process merely a sub-selection of the pressure readings and/or frames I. To this effect, an optional sampler SP may be used that samples the image feed and/or pressure reading feed to produce by sub-selection a sub-set of the originally received feeds I,P at input port(s) IN, and it is this subset that is then processed by the system SYS to compute the prediction result R.

Various time- or event-driven sampling schemes may be implemented by the sampler SP. For example, the sampler SP may sample more densely the more recent images and/or pressure readings, and decrease the sampling frequency over time. Preferably, it is ensured that an initial image from the beginning of the balloon inflation phase is always included. A number of different events could be used for indicating the beginning of the balloon inflation phase. For example, the said beginning could be taken as the time (T) at which inflation volume is less than a certain volume threshold, such as less than 5 mL. Alternatively, the said time T is one where pressure is at a certain threshold, such as at 0 atm. Alternatively still, the said time T may be image-defined, for example as the time T at which no balloon footprint is visible in the image feed.

With continued reference to variable sampling frequency schemes, the sampling may proceed evenly over a certain period of time. More dense sampling (higher sampling frequency) may be used to sample most recent time points and then decrease the sampling frequency over time. As a further variant, the sampling is done only when there is a minimum difference between subsequent images and/or corresponding pressure readings. The various sampling schemes and definition of start time T may be used for sampling the imagery and/or the pressure readings. Preferably the same sampling schemes may be use for both, the image channel and the pressure reading channel, although the schemes may differ. For example, if there are no changes in channel, sampling may pause whilst sampling in the other channel may be ongoing. To maintain pairing or synchrony between the two channels, copies of a constant image or pressure reading may be used for pairing up with values in the channel that is being sampled.

In the following, unless specific reference is made to the sampler SP, we shall not distinguish whether the whole or a sampled portion of the imagery or pressure readings are processed.

The images I and the pressure readings P are processed by the machine learning module MLM. As briefly mentioned above, the machine learning module MLM may include in embodiments a pre-trained machine learning model M. The machine learning model M is used to process the input data, that is, the image(s) and pressure reading(s) I,P. We shall refer in the following to the images and the pressure reading feeds as the main input or simply the input data and denote this as “(I,P)”.

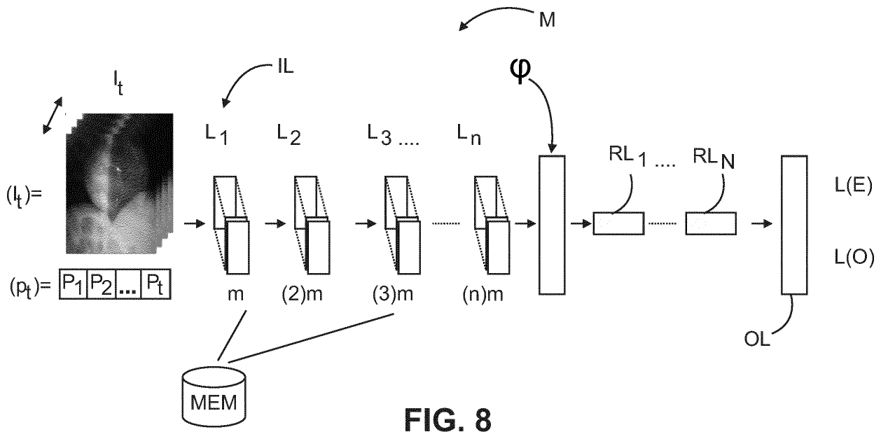

The machine learning model M includes a set of parameters which can be adjusted by a machine learning algorithm based on the training data. The machine learning model is hence operable in two modes in training mode and in deployment mode, as will be explained in more detail below at FIGS. 8-10.

Whilst model-based ML techniques are mainly envisaged herein for machine learning from the input data (I,P), such model-based approaches are not a necessity herein, and non-model based machine learning techniques/algorithms are specifically envisaged herein in alternative embodiments.

In deployment, the pre-trained machine learning model M processes the input data (I,P) and produces the desired result R. The result R may includes data E indicative of whether an adverse effect such as a balloon rupture is imminent. In addition or instead, the indication provided by the result R may be one of a medical outcome O to be expected in relation to the patient and/or the performed intervention at the given site ST.

A communication network CN may be used to forward in particular the results R to a storage MEM-R or to the (or other) display device DD for display. The results may also be forwarded through the communication network to any other processing component as desired.

The computed result R may be suitably processed by a reporting module RM. Such processing may include graphical or textual rendering, or a combination thereof. To this end, the reporting module RM may include the visualizer component VC which allows displaying the predicted data R on the display device DD. The original input data (I,P) may be displayed in full or in parts alongside the result R. The reporting module RM may be operable to store all or parts of the computed results R in the result storage MEM-R. The reporting module RM may be operable in an alert mode where, once the predicted results indicate that an adverse event is imminent, the acquired data (I,P) is auto-stored in a data storage such as the result storage MEM-R or other data memory.

An annotation tool AT may be used to annotate at least a portion of currently acquired imagery. Whether or not the annotation is applied is a function of the predicted result R.

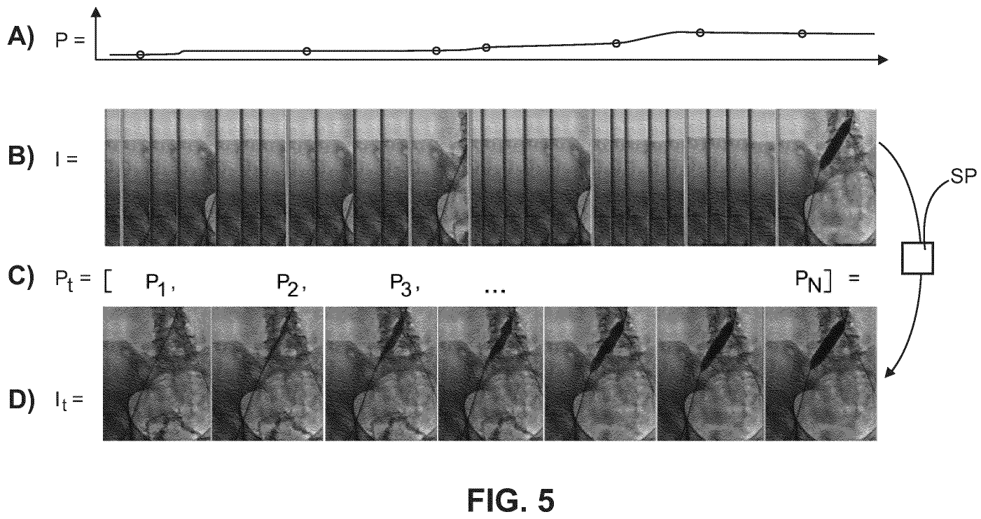

FIG. 5 shows a schematic representation of input data (I,P) with row A) showing a time series of acquired pressure readings P whilst row B) shows a time series of images I acquired by the imaging apparatus IA during the intervention. Rows C), D) are respective sampled data sets produced by the sampler SP. Specifically, row C) shows the sampled pressure readings Pₜ at discrete time intervals t running from I through N, and Iₜ showing the respectively sampled images I at corresponding times t.

In general, the input data (I,P), sampled or not, comprises corresponding pairs of images Iₜ, and pressure readings Pₜ. Data components Iₜ and Pₜ can be said to be paired because they represent imagery and pressure readings, both at the same respective, given time t. The above mentioned time stamping may be used to establish sets of pairs. The described pairing via time-stamping or otherwise is optional and the proposed machine learning MLM may also be able to process unpaired readings (I,P), such as when there is too much noise in the data. However, the time-based pairing of the input data feeds is preferred as this may accelerate the learning.

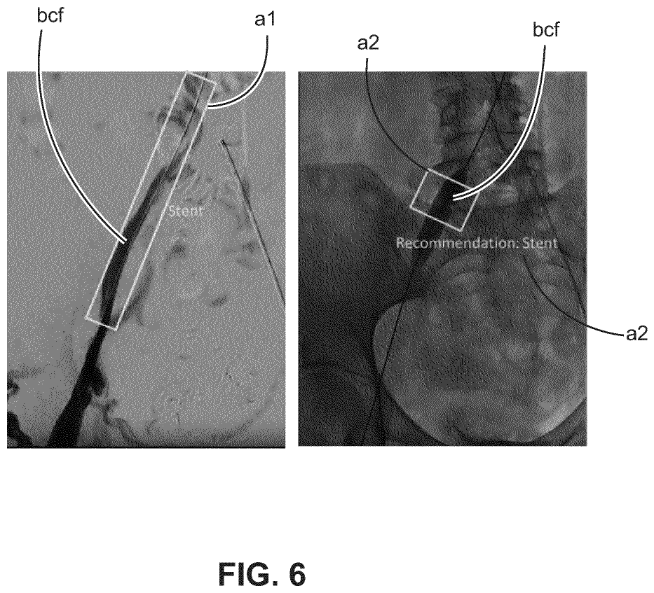

Reference is now made to FIG. 6A, B which illustrate operation of the previously mentioned annotator AT. In the example of FIG. 6A), there is an annotation a1 in relation to the balloon catheter footprint bcf. A similar annotation a2 is shown in FIG. 6B). The annotation may be purely graphical as shown by the rectangles in FIG. 6. Other shapes such as circles ellipses, triangles or others still may be used instead or in addition. The graphical annotations a1, a2 may be augmented by additional information such as textual information. For example, the annotations may represent text components to suggest follow-up treatments, such as the shown example “recommendation:stent”. The predicted results may be combined with angiographic data for example, preferably (immediately) following ballooning. At that point, the user and/or the annotator AT may then annotate a1, a2 any regions of the lesion ST that may require further treatment, and which kind of treatment, prior to proceeding with that additional therapy (e.g. atherectomy, stenting, drug-coated balloon, etc.). The annotation operation is preferably an auto-annotation and is triggered in response to the probability of the adverse event. For example, if the machine learning module MLM returns a probability value in excess of a probability threshold, eg p=10%, auto-annotations can be triggered. Whilst the annotator itself may be implemented in machine learning, this may not be required in all embodiments and the recommended clinical actions encoded in the annotations may be retrieved by look-up operation from medical knowledge databases, or are added manually by the user. If ML-based annotation is used, the training data may include such training annotations associated with the image-pressure reading training pairs.

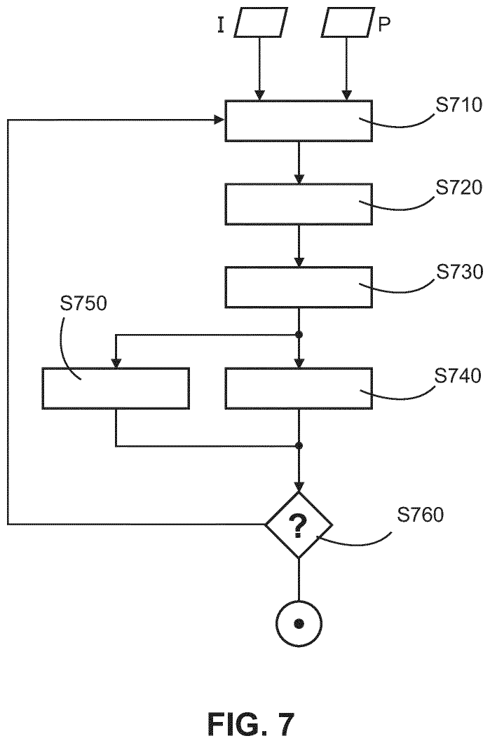

Reference is now made to the flow chart in FIG. 7 which shows steps of a computer implemented method that may be used to implement the machine learning based system SYS for support of interventional medical procedures, in particular those involving balloon catheters. The method described in the following may be understood to implement the above described computerized system SYS, but the steps may also be understood as a teaching in its own right and are hence not necessarily tied to the system architectures discussed above.

In the following, when referring to the method steps in FIG. 7, these are carried out during deployment of the machine learning module. In other words, when referring to FIG. 7, it is assumed that the machine learning module has already been at least partially pre-trained in a suitable training procedure, based on training data. The training procedure or training phase will be described more fully below at FIGS. 8-10.

Turning now in more detail to the method, at step S710 the input data (I,P) is received, not necessarily simultaneously. The input data comprises one or more images acquired by the imaging apparatus during the intervention using a medical device such as balloon catheter, and one or more pressure readings acquired by a pressure sensor. The sensor is advantageously in-situ, such as integrated into the interventional device, such as in the balloon catheter. The sensor may be integrated in the balloon portion of the balloon catheter, to measure the pressure inside the balloon as this is inflated or deflated and/or whilst the balloon interacts with the vessel walls. The balloon may be deformed such as twisted and kinked during the inflation or deflation. The balloon portion may fold-over. Such interactions may thus cause higher than expected pressure readings or larger pressure changes such as pressure spikes or drops. The sensor may supply the readings through a wired or wireless connection to one or more data processing units PU, such as computing devices with one or more suitable processors that are configured to implement the method at least in parts.

Preferably, the input data are paired so that the balloon portion FP configuration as captured in a given frame at a given time instant corresponds to the pressure reading obtained at substantially the same time instant by the pressure sensor S. The frames I and pressure readings P are either natively paired or may be processed, for example by time-stamping matching, in an optional step to arrange the input data into paired form. However, having the input data paired is not a necessity herein, but may be beneficial as mentioned earlier.

In an optional step S720 the feed of frames I and/or the pressure reading feed P in the input data (I,P) are sampled according to a sample scheme as described above.

The so sampled input data (I,P) or the whole of the input data (I,P) is then processed by the machine learning module at step S730 to predict the result R.

The result R may be a classification or regression result. In particular, the input data at step S730 is classified as indicative of an adverse event in relation to the patient and/or the interventional device, such as the balloon catheter. In particular, the event may describe the likelihood/probability for a balloon rupture to occur. In addition or instead, the result R is classified or regressed into an indication of a medical outcome in relation to the patient undergoing the procedure as captured by the pressure readings and the images received in the input data (I,P) at step S710.

At step S740 the prediction result(s) R is/are output.

The outputting step S740 may include a reporting step in which the predicted result is transformed into suitable format such as textual, number or graphical, or into a hybrid format including any two or more of text, numbers and graphics. The raw results or the result in reportable form may be forwarded through a communication channel to a recipient, such as a communication endpoint, a user device such as a mobile phone, mobile computer, a desktop computer or any other computing device.

At an optional step S750 the input data (I,P) is stored at least in parts in a data storage as a function of the prediction result R. For example, and mainly envisaged herein in embodiments, if the result R indicates that an adverse event, such as the balloon rupture event, is imminent, an auto-save or storing function is initiated that causes the currently received imagery and/or pressure readings to be stored. Such storing operation continues for a pre-defined and/or user defined period of time after the result indicative for such an adverse event has been computed. This allows capturing data that may then be used by manufacturers or other interested parties to precisely understand and analyze the malfunction situation, if indeed a rupture did occur. Specifically, the triggering of the auto-saving of interventional x-ray and pressure data may optionally depend on the computed likelihoods. The auto-save operation may be triggered when an adverse event likelihood exceeds a threshold such as p=10%, or any other such threshold as may be defined by the user.

The reporting step in particular may also include annotating the currently received imagery and/or pressure readings, as function of the computed result, as has been described above in relation to FIG. 6.

At step S760 it is then checked whether new input data is received at one or more input ports, and, if yes, the method then repeats at step S710 and proceeds as described above. If no more data is received the procedure terminates. The method may operate at suitable processing frequency per single frame and/or single pressure reading datum. Alternatively and preferably, as mentioned above, in particular in order to account for trends or for saving computational resources, the method may operate in blocks. Once a block of a predefined number or plural frames and/or plural pressure readings are received, the machine learning based computing of the result at step S730 is based on processing the blocks of frames and/or pressure readings at once, jointly. The block size for the processable frames or pressure readings may be the same (such as in paired data) or may differ. In some embodiments, a single frame is processed with a block of plural pressure readings, or a single pressure reading may be processed with a block of plural frames. Preferably however, a plural block of frames is processed together with a block of pressure readings of the same or different size.

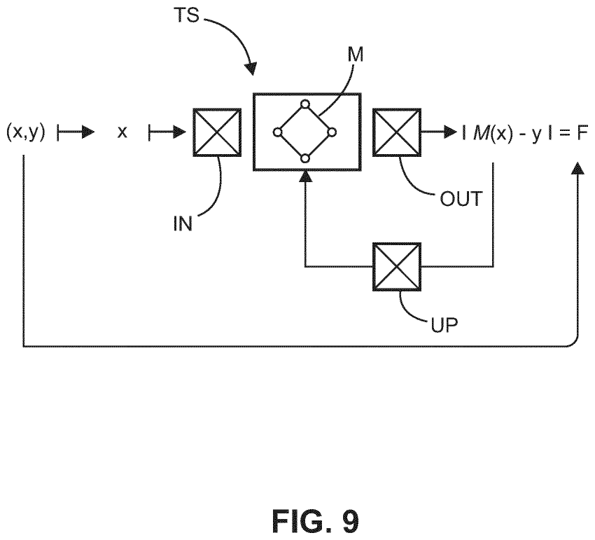

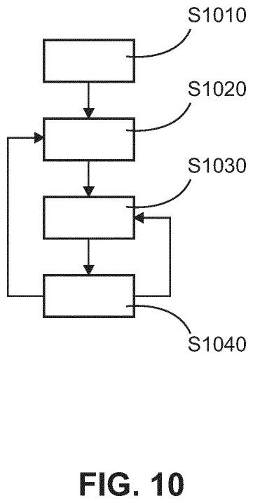

As indicate above, the following will provide more details on the machine learning implementation of the above described method and/or computerized system SYS. Broadly, FIG. 8 will provide more details of a machine learning model that is envisaged in embodiments. FIGS. 9 and 10 will then describe in more detail the training aspects to train a machine learning model, such as the one in FIG. 8, but also others.

Turning now first to FIG. 8, this shows a schematic block diagram of a neural network based model M envisaged herein. Specifically, in embodiments, but not necessarily in all embodiments, a neural-network (“NN”) type model may be used. In particular, and in embodiments, an at least partially convolutional neural-network type (“CNN”) is used, which includes one or more layers that are non-fully connected layers.

The model M may be trained by a computerized training systems TS to be described more fully below at FIG. 9. In training, the training system TS adapts an initial set of (model) parameters θ of the model M. In the context of neural network models, the parameters are sometime referred to herein as weights. The training data may be generated by simulation or may be procured from existing historic imagery or other data as may be found in medical image database such as in PACS (picture archiving and communication system) or similar as will be described in more detail below in relation to FIG. 9. Two processing phases may thus be defined in relation to the machine learning model M: a training phase and a deployment (or inference) phase. In training phase, prior to deployment phase, the model is trained by adapting its parameters based on the training data. Once trained, the model may be used in deployment phase to predict a probability for the mentioned adverse events, such as for a balloon rupture event, and/or a probability of a medical outcome as mentioned earlier. The training may be a one-off operation, or may be repeated once new training data become available. Training and deployment may thus occur interleaved phases in embodiments.

The machine learning model M may be stored in one (or more) computer memories MEM. The pre-trained model M may be deployed as a machine learning component MLM that may be run on a computing device PU, such as a desktop computer, a workstation, a laptop, etc or plural such devices in a distributed computing architecture. Preferably, to achieve good throughput, the computing device PU includes one or more processors (CPU) that support parallel computing, such as those of multi-core design. In embodiments, GPU(s) (graphical processing units) or one or more TPUs (tensor processing unit) are used.

Referring now in more detail to FIG. 8, the network M may comprise a plurality of computational nodes arranged in layers Lᵢ, RLⱼ in a cascaded fashion, with data flow proceeding from left to right and thus from layer to layer in some feedforward layers Li. One or more recurrent layers RLⱼ ma be used in addition (as shown) or instead.

In training, the input data x=(I′,P′) is applied to input layer IL. The input data x then propagates through a sequence of hidden layers Li, RLⱼ(there may be more or less than shown in FIG. 8), to then emerge at an output layer OL as an estimate output M(x)=R′, an estimate for the mentioned result L(E) or L(O). The output data, being mainly a classification or regression result, has usually a different size than the input date x. In the following, a tilde notion “′” may be used to indicate data applied or generated during training such as the training imagery I′ and the training pressure readings P′ or the estimated result R′, to distinguish from data used/generated during deployment, denoted as (I,P) and R as has been done earlier.

The model network M may be said to have a deep architecture because it has more than one hidden layer. In a feed-forward network, the “depth” is the number of hidden layers between input layer IL and output layer OL, whilst in recurrent networks the depth is the number of hidden layers, times the number of passes. In recurrent layers, at least part of their output is fed back to earlier layers, such as the immediately preceding layer. No such feeding back occurs in feedforward type layers.

The layers of the network, and indeed the input (I′,P′) and output R′, and the intermediate input and output between hidden layers (referred to herein as feature maps), can be represented as vectors, or as two or higher dimensional matrices (“tensors”) for computational and memory allocation efficiency. The dimension and/or the number of entries represent the above mentioned size.

The input layer IL of the neural network model M is suitably dimensioned to receive, in training phase, the training input data, referred to herein as (I′, P′). The processing may proceed frame by per frame and/or per individual pressure reading datum P′ₜ. Alternatively, the processing is in blocks of frames or pressure readings, each blocks comprising plural frames or pressure readings. A reference to block-wise processing as described above is understood to include the degenerated case of blocks of length of length 1. If required, we will refer to blocks of length 1 or greater than 1, if required and to emphasize that more than one frame or pressure reading datum is processed at once.

The frames of pressure reading blocks are preferably applied simultaneously to the input layer OL, but sequential processing is not excluded herein. The block of pressure readings may be represented as an image volume, with one dimension indicating time, each time coordinate referring to a specific frame. The block of pressure readings may be represented as a vector of suitable length defining the pressure reading block length, as shown in FIG. 8. In the following, we will no longer distinguish between per frame/pre pressure reading datum as the block length for both data types, imagery and pressure readings, is understood to be 1 or larger.

The processing of the training input data (I′,P′) may be implemented by processing the pressure readings together with the imagery, or each block may be separately processed in different sequences of layers. Intermediate results of the pressure reading processing layers and the image processing layers may then be merged and processed together in one or more subsequent layers.

The embodiment in FIG. 8 shows an architecture where both the images and the pressure readings are processed together at each layer but as said this may not necessarily be the case in all embodiments, and processing may occur in separate strands or sequences of such layers. In embodiments, parts of the network M may be arranged as a transformer or shaper network that transforms the pressure readings into pseudo-images. The pseudo-images are then feed into feature maps of the layers processing the “real imagery” I′. Pseudo-images and real images may be processed together as channels of a multidimensional image volume with a “pressure reading channel”. Specially, in embodiments there are initially two inputs that have separate input branches (eg, convolutional and/or fully-connected layers) and a concatenation of the inputs occurs at the level of the feature maps. Such a concatenation may require reshaping/reformatting the image and/or pressure readings inputs by using various operations such as tailing, spatial and temporal pooling, 1×1 convolutional operations, etc. For example, the pressure values may be copied multiple times to create a matrix that has a similar size as the input image (e.g. an n×n pseudo-image) and is then incorporated into the input IN as a separate channel, or via various algebraic operations, such as multiplication, addition, division, etc.

As mentioned earlier, the model M may include one or more hidden layers. As shown in FIG. 8, the layers may include one or more convolutional layers indicated herein as Lᵢ, for example i=1 . . . n. The first such layer L1 is the input layer IL where the input data is received and processed at that layer. The layers are preferably of the feed-forward type. Each convolutional layer Li comprises a certain number of filters. The number of filters is in general growing as one progresses through the layers. However, other embodiments are also possible, such as maintaining the number of filters with network depth, or by decreasing the number of filters along network depth. For example, in embodiments where the filter number is changing, the said filter element number may grow or decrease linearly with layer position. For example, suppose layer Lⱼ includes m filters, then Layer Lⱼ₊₁ may include (j+1) times m filters, etc. Instead of convolutional feed-forward layers, one or more fully-connected feedforward layers may be used. Alternatively, the feedforward layers may include a mix of convolutional and fully connected layers. In embodiments, at least one, more than one, or each layer L, may comprise one or more, or a combination of, convolutional operation, batch normalization, dropout, pooling, and non-linearity operation.

Upstream or downstream the sequence of feedforward convolutional layers, there are one or more optional recurrent layers RLⱼ, example, j=1 . . . N. The recurrent layers may be convolutional or fully connected, or may include a mix of convolutional and fully connected layers.

Recurrent type layers have been found to be beneficial when processing time dependent data, such as the pressure readings and/or the image frames in the video feed. Convolutional layers have been found to be beneficial in processing image type data. If the data is not image-like, that is, there are no spatial correlations, such as in the vector of pressure readings, one or more fully connected layers of the recurrent or feedforward type may be used. Preferably, the layers are of the recurrent type.

With more detailed reference to the embodiment shown in FIG. 8, one, or preferably more, convolutional layers Lᵢ regress or classify the received training data input (I′,P′) into one or more feature maps or feature vectors ϕ. The feature maps/vectors ϕ is/are then passed on to be processed by a sequence of one or more sub-sequent recurrent layers RLⱼ. Additional functional layers such as dropout layers, batch normalization, convolutional layer, spatial pooling (max, average, min), global pooling, may be used.

An output layer OL processes the received feature maps from the previous one or more layers (such as recurrent layers RLⱼ) into a training estimate for the output result, denoted herein as M(I,P)=R′. That is, the ML model M with parameters θ are applied to training data (I′,P′), imagery and pressure readings from the training data, to yield training result R′. The output layer OL may implement a regression or classification as desired. In embodiments, the output result R′ represents the probability P(E) of the adverse event in relation to the balloon catheter for the observed input data I′,P′. Alternatively, or in addition, the result R′ may represent a probability P(O) of a medical outcome in relation to the treated patient.

Once trained, the parameters θ have been suitably adjusted and are now fixed as θ′. In the described neural network model, the adjusted parameters θ′ include in particular the filter elements of layers RLᵢ,Lⱼ. The processing by the trained network M^{θ}′ of “real”, unseen-before input data (I,P) during deployment in clinical practice, is exactly as has been described above in relation to FIG. 8 during training, only that now non-training input data (I,P) is used in place of training data (I′,P′) to obtain result R in place of training result R′. Specifically, if the model M^{θ}′ is of the neural network type, the input data is applied to the input layer IL to be then propagated through the layers to then emerge as result R at output layer OL. The nature of the processing may differ for other models but it will be understood that the above described principles in relation to ML model M^{θ}′ still apply.

In some embodiments, output layer OL of model M is configured for classification, such as multi-label or binary classification. For example, the result R,R′ of interest is whether or not a rupture event or other adverse effect is to occur. In this embodiment, the output R,R′ is classified into a vector with two entries indicating whether or not there is such an event. As a further variant instead of such a binary output, a probability distribution over more than two entries may be computed where the entries indicate whether or not there is a probability for a particular event. In an extreme case only a single number is provided at the output result, the output being an indication for an event that attracted the highest probability. It is in particular in such classification setups that the model M may include, in addition to the one or more convolutional layers IL, L₁-L_{N}, one or more fully connected layers, of the feedforward or recurrent type. In case of a classification result, the output layer OL may be configured as a softmax-function layer or as similar computational nodes where feature maps from previous layer(s) are combined into normalized counts to represent the classification probability per class.

Whilst classification is mainly envisaged herein, regression is not excluded and indeed envisaged in embodiments, in particular in cases where the pressure sensor S is arranged to measure the pressure distribution across the balloon. For example, a pressure sensor patch may be used to infer a pressure distribution. A FORS (Fiber optic real shape) fiber or any other type of fiber optical shape sensing may be used. In such embodiments the pressure reading Pₜ is a 2D or 3D array rather than a scalar. The output layer OL may thus operate to regress the pressure distribution into probability maps rather than a single output value. The probability maps may be displayed by visualizer VC on display device DD. The results R on adverse events may hence be localized to indicate where the balloon portion FP may rupture, twist or unfold, rather than merely providing a single probability value of the event as described above for a pure classification task. Similarly, a medical outcome may be localized to indicate where the restenosis may originate.

As mentioned earlier, the preprocessor PP may segment the imagery for regions of interest. This option may be used to limit the dimensionality of the input space. The segmentations for the balloon or vessel tree may be provided as additional input to the machine learning component. For example, the segmentations may represent the earlier mentioned additional contextual parametric descriptions for both training and inference, instead of (or in addition to) providing the raw images I. If the segmentations or other image pre-processing data is used as additional input, these may be processed by the model M as additional image channels, the true image data and the additional input forming image volumes, with one or more dimensions used to define the channels, similar to the manner which the pressure readings may be processed as described above after transformation into pseudo-imagery. Rather than using the data by the preprocessor as additional information, the preprocessed image data may be processed by the machine learning component instead of the raw image data.

It will be understood that the above described model M in FIG. 8 is merely according to one embodiment and is not limiting to the present disclosure. Other neural network architectures are also envisaged herein with more or less or different functionalities than described herein, such as drop-out layers mentioned above, or pooling layers or others still. Multilayer perceptron (MLP) type NNs are also envisaged in alternative embodiments. What is more, models M envisaged herein are not necessarily of the neural network type at all, such as SVMs, decision trees (eg, random forests). Other, classical statistical regression or classification methods based on sampling from training data are also envisaged herein in alternative embodiments such as multivariate regression (weighted linear or logistic regression). Still other techniques may include Bayesian networks, or random fields, such as Markov type random field and others.

Reference is now made to FIG. 9 which shows more details of a computer-based training system TS that may be used to train the machine learning model M^{θ} such as the one described in FIG. 8 or other. However, using a model-based approach is not necessary herein and non-model based “free” machine learning approaches are also envisaged herein. If a model based approached is used, the neural network type model such as the one described above in relation FIG. 8, whilst envisaged herein in the main, are not at the exclusion of other machine learning techniques, such as support vector machines and others.

The training system TS may be used for adjusting training the parameters, for example the weights of a neural network-type model as discussed in FIG. 9 or other neural network-type model, or indeed non-neural network type ML models. The training system TS may be used for adjusting weights of neural network-type machine learning model such discussed above in connection with FIG. 8. The weight may include parameters of the filter elements. The weights of each filter are a set of numbers used to compute the convolution at the given layer depth. In convolutional layers, the filters are used to compute an entry of an output feature map from a sub-set of entries of a previous feature map. In general, the sub-set differs for each output entry. In a fully-connected layer, such an entry of an output feature map is computed in general from all entries of the previous feature map.

In general, the training is based on training data. Training data comprises k pairs of data items (xₖ, yₖ). index k may run into the 100s or 1000s. The training data comprises for each pair k, training input data xₖ and an associated target yₖ. For present purposes, xₖ=(Iₖ′,Pₖ′) and yₖ=Rₖ′, with Iₖ′ indicating test imagery (one or more images) for the k-th pair, and P′ₖ indicates the associated pressure reading block. The training result or label Rₖ′ indicates an adverse event or medical outcome, as desired. The training data may thus be organized in k pairs in particular for supervised learning schemes as mainly envisaged herein. However, it should be noted that non-supervised learning schemes are not excluded herein.

Some or each training data pair k may be stored as a 3-tuple:

dₖ=(xₖ,yₖ)=(I′ₜₖ,P′ₜₖ,R′ₜₖ) (1)

Given such pair, random batches of training instances may defined.

In (1) and for some angioplasty embodiments, I′ₜᵢ represents the sampled time-series 2D fluoroscopy projection acquired during the interventional procedure during balloon inflation. P′ₜᵢ represents the sampled time-series pressure measurement acquired during the interventional procedure during balloon inflation. R′ᵢ represents the trial or registry data that is recorded for that patient including. The trial or registry data may encode the adverse event, such as balloon rupture, balloon folding. The result R′ᵢ may represent instead or addition, patency at m months post-procedure (eg, m=6, 12, 18 or other) or freedom from device and procedure-related death, or any other. If the model M is to be trained for localized results in form or probability maps etc the target yᵢ=R′ᵢ may need to be preprocessed using other machine learning techniques for more advantageous formatting, for example to reduce sparsity. One-hot encoding may be used which may be optionally transformed into another representation (sometimes referred to as “code”) by an auto-encoder for example.

The training input data xₖ may be obtained from historical image data acquired in the cath lab and held in image repositories, such as the PACS of a HIS (hospital information system) for instance. The pressure readings may likewise be found in medical databases. The targets yₖ or “ground truth” represents labels of interest for classification type models M, and is an indication for the mentioned balloon rupture or other adverse events. Medical outcomes may be sourced from medical health records of former cath lab patients. The respective labels may be found in header data or could be inferred from inspecting the medical records and notes. Textual reports on historical balloon catheter interventions may be required by a search engine for keyword tell-tales such as “rupture”, “compromised balloon” etc. a. There are large datasets available for clinical trials and registries that track outcomes in relation to new therapy devices, in particular for drug-coated balloons. In such registries, time-series of x-ray and corresponding pressure measurements are stored, as well as trial/registry reporting values. Preferably the training data set is sourced from a large and variable patient population (as is normally reflected in registries). The patient population from which the training data is sourced preferably includes patients with varying anatomical features, disease severity, disease location, gender, age, and medical history.

In the training phase, an architecture of a machine learning model M, such as the shown NN-type of FIG. 8 is pre-populated with an initial set of weights. The weights θ of the model represent a parameterization M^{θ}, and it is the object of the training system TS to optimize and hence adjust the parameters θ based on the training data (xₖ, yₖ) pairs. In other words, the learning can be formulized mathematically as an optimization scheme where a cost function F is minimized although the dual formulation of maximizing a utility function may be used instead.

Assuming for now the paradigm of a cost function F, this measures the aggregated residue(s), that is, the error incurred between data estimated by the model M and the targets as per some or all of the training data pairs k in a batch:

argmin_{θ}F=Σₖ∥M^{θ}(xₖ),yₖ∥ (2)

In eq. (2), function MO denotes the result of the model M applied to training input x for a given training pair k. The cost function may be a (squared) Euclidean-distance type cost function (such as least squares or similar) preferably used for regression tasks. However, for classifier tasks as envisaged herein, the summation in (2) is formulated instead as one of cross-entropy or Kullback-Leiber divergence or similar.

In training, the training input data xₖ of a training pair is propagated through the initialized network M. Specifically, the training input xₖ for a k-th pair is received at input IL, passed through the model and is then output at output OL as output training data M^{θ} (x). As indicated, a suitable measure ∥•∥ is used such as p-norm, squared differences, or cross-entropy, to measure the difference, also referred to herein as residue, between the actual training output M^{θ} (xₖ) produced by the model M, and the desired target yₖ.

The output training data M(xₖ) is an estimate for target yₖ associated with the applied input training image data xₖ. In general, there is an error between this output M(xₖ) and the associated target yₖ of the presently considered k-th pair. An optimization scheme such as backward/forward propagation or other gradient based methods may then be used to adapt the parameters θ of the model M so as to decrease the residue for the considered pair (xₖ, yₖ) or a subset of training pairs from the full training data set.

After one or more iterations in a first, inner, loop in which the parameters θ of the model are updated by updater UP for the current pair (xₖ,yₖ), the training system TS enters a second, an outer, loop where a next training data pair x^{k+1}, y^{k+1} is processed accordingly. The structure of updater UP depends on the optimization scheme used. For example, the inner loop as administered by updater UP may be implemented by one or more forward and backward passes in a forward/backpropagation algorithm. While adjusting the parameters, the aggregated, for example summed, residues of all the training pairs are considered up to the current pair, to improve the objective function F. The aggregated residue can be formed by configuring the objective function F as a sum of residues such as in eq. (2) of some or all considered residues for each pair. In the inner loop, more than one pair may be provided to minimize (2) at once, the number of so provided pairs often referred to as batches of training data. The optimization in the outer loop than progressed from batch to batch until all training data, or a pre-defined minimum number, has been exhausted. It may also be possible to process all training data at once although in most cases this will be inefficient and progression in subsets of training data (that is, the batches) may be preferable.

The training system as shown in FIG. 9 can be used for all learning schemes, in particular supervised schemes. Unsupervised learning schemes may also be envisaged herein in alternative embodiments. GPUs may be used to implement the training system TS, in particular for NN-type models M.

The fully trained machine learning module M may be stored in one or more memories MEM or databases, and can be made available as pre-trained machine learning models for use in system SYS. The trained model M may be made available in a cloud service. Access can either be offered free of charge or their use can be granted via license-pay or pay-per-use scheme.

Reference is now made to FIG. 10 which shows a flow chart of a computer-implemented method of training a machine learning model, based on training data as may be used to implement the above described training system TS or others.

Suitable training data needs to be collated from historic intervention, such as balloon catheterizations, and as documented in medical databases mentioned above. Preferably, supervised learning schemes are envisaged herein although this is not a necessity as unsupervised learning setups are also envisaged herein.

In supervised learning, the training data includes suitable pairs of data items, each pair including training input data and associated therewith a target training output data. Specifically, the pairs comprise imagery and pressure readings suitably labeled in relation to adverse events and/or medical outcome. The imagery and pressure readings can be paired up with their result label by retrieving the same from historic balloon catheterization treatment data records, health records or other medical data repositories, as described above.

With continued reference to FIG. 10, at step S1010 training data is received in the form of one or more pairs (xₖ,yₖ), depending one batch size. Each pair includes the training input xₖ and the associated target yₖ. xₖ, as defined in FIG. 7 above. The batch size is different from the above mentioned block-size. The block-size determines the number of images and/or pressure readings per training pair, whilst the batch-size determines the number of pairs to processed at once when minimizing cost at (2) or otherwise improving cost function F. Batch size could be 1, 8, 16 . . . 256, or any other number, or even the full dataset. In the first example we train the model using stochastic gradient descent (1), or mini-batch gradient descent, or batch gradient descent.

At step S1020, the training input xₖ is applied to an initialized machine learning model NN to produce a training output yₖ.

At step S1030 a deviation, or residue, of the training output M(xₖ) from the associated target yₖ is quantified by a cost function F. One or more parameters of the model are adapted at step S1040 in one or more iterations in an inner loop to improve the cost function. For instance, the model parameters are adapted to decrease residues as measured by the cost function. The parameters may include in particular weights W of each node in each layer.

The training method then returns in an outer loop to step S1010 where the next one or more pairs of training data (depending on batch size) is fed in. In step S1020, the parameters of the model are adapted so that the aggregated residues of all pairs considered are decreased, in particular minimized. The cost function quantifies the aggregated residues. Forward-backward propagation or similar gradient-based techniques may be used in the inner loop.

More generally, the parameters of the model M are adjusted to improve objective function F which is either a cost function (see eq (2) above) or a utility function. In embodiments, the cost function is configured to the measure the aggregated residues. In embodiments the aggregation of residues is implemented by summation over all or some residues for all pairs considered, depending on batch size. The method may be implemented on one or more general-purpose processing units TS, preferably having processors capable for parallel processing to speed up the training.

Whilst the system SYS for intervention support and the training system have been explained above with main reference to x-ray imaging, other imaging modalities such as 4D CT/MRI, ultrasound, OCT (optical coherence tomography) or any other times series based imaging are also envisaged herein. Some imaging modalities such as OCT could be integrated in the balloon catheter itself. Endoscopic imaging is also envisaged herein.

The components of system SYS,TS may be implemented as one or more software modules, run on one or more general-purpose processing units PU such as a workstation associated with the imager IA, or on a server computer associated with a group of imagers.

Alternatively, some or all components of the system TS, SYS may be arranged in hardware such as a suitably programmed microcontroller or microprocessor, such an FPGA (field-programmable-gate-array) or as a hardwired IC chip, an application specific integrated circuitry (ASIC), integrated into the imaging system IA. In a further embodiment still, any one of systems TS, SYS may be implemented in both, partly in software and partly in hardware.

The different components of any one or system TS, SYS may be implemented on a single data processing unit PU. Alternatively, some or more components are implemented on different processing units PU, possibly remotely arranged in a distributed architecture and connectable in a suitable communication network such as in a cloud setting or client-server setup, etc.

One or more features described herein can be configured or implemented as or with circuitry encoded within a computer-readable medium, and/or combinations thereof. Circuitry may include discrete and/or integrated circuitry, a system-on-a-chip (SOC), and combinations thereof, a machine, a computer system, a processor and memory, a computer program.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above-described apparatus. The computing unit can be adapted to operate automatically and/or to execute the orders of a user. A computer program may be loaded into a working memory of a data processor. The data processor may thus be equipped to carry out the method of the invention. This exemplary embodiment of the invention covers both, a computer program that right from the beginning uses the invention and a computer program that by means of an up-date turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium (in particular, but not necessarily, a non-transitory medium), such as an optical storage medium or a solid-state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A system for supporting a balloon catheter procedure, the system comprising:
at least one input interface configured to receive input data, comprising i) image data acquired of a balloon catheter in a vessel of a patient, and ii) one or more pressure readings collected by a pressure sensor of the balloon catheter;
a processor configured to predict, based on the input data, a prediction result including an event in relation to at least one of i) the balloon catheter and ii) a section of a vessel in which the balloon catheter is residable.

2. The system of claim 1, wherein at least one of the image data and the one or more pressure readings is obtained by sampling a respective original data stream, wherein a frequency of the sampling is variable.

3. The system of claim 1, wherein the processor is further configured to report the prediction result at least one of numerically and/of graphically.

4. The system of claim 3, wherein the processor is further configured to produce a graphics display for displaying the prediction result on a display device.

5. The system of claim 4, wherein the graphics display further includes at least a portion of the image data and the one or more pressure readings.

6. The system of claim 3, the processor is further configured to annotate at least a portion of the received image data with the prediction result.

7. The system of claim 1, wherein, based on the prediction result, the processor is configured to store at least a portion of the image data and the one or more pressure readings in a memory.

8. The system of claim 1, wherein the processor applies a trained machine learning model of the neural network type, the trained machine learning model configured to predict the prediction result.

9. The system of claim 8, wherein the trained machine learning model includes at least one of a convolutional layer and a recurrent layer.

10. The system of claim 1, wherein the event is a rupture or twisting of a balloon portion of the balloon catheter, or stress on the vessel; caused by an inflation of the balloon.

11. The system of claim 1, further comprising a processor configured to train, based on training data, a machine learning model to predict the prediction result.

12. A method of supporting a balloon catheter procedure, the method comprising:
receiving input data comprising i) image data acquired of a balloon catheter in a vessel of a patient and ii) one or more pressure readings collected by a pressure sensor of the balloon catheter; and
processing the input data by a trained machine learning model to obtain a prediction result for an event in relation to at least one of i) the balloon catheter and ii) a section of a vessel in which the balloon catheter is residable.

13. The method of claim 12, further comprising training, based on training data, the machine learning model.

14. A non-transitory computer-readable storage medium having stored a program comprising instructions, which, when being executed by a processor, causes the processor to:
receive input data comprising i) image data acquired of a balloon catheter in a vessel of a patient and ii) one or more pressure readings collected by a pressure sensor of the balloon catheter; and
process the input data, by a trained machine learning model, to obtain a prediction result for an event in relation to at least one of i) the balloon catheter and ii) a section of a vessel in which the balloon catheter is residable.

15. (canceled)
